(19) United States
(12) Patent Application Publication
(10) Pub. No.: US 2018/0289332 A1
(43) Pub. Date: Oct. 11, 2018

(54) SENSOR SYSTEM, SENSOR INFORMATION PROCESSING APPARATUS, NON-TRANSITORY COMPUTER-READABLE RECORDING MEDIUM HAVING STORED THEREIN SENSOR INFORMATION PROCESSING PROGRAM, AND BED

(71) Applicant: FUJITSU LIMITED, Kawasaki-shi (JP)

(72) Inventors: TAKAYUKI YAMAJI, Yokohama (JP); Yuta MASUDA, Kawasaki (JP)

(73) Assignee: FUJITSU LIMITED, Kawasaki-shi (JP)

(21) Appl. No.: 16/003,650

(22) Filed: Jun. 8, 2018

Related U.S. Application Data
(63) Continuation of application No. PCT/JP2015/084558, filed on Dec. 9, 2015.

(51) Int. Cl. A61B 5/00; G06K 9/00; A61B 5/11; A61B 5/0205; A61B 5/05; G01S 13/50; G01S 13/87

(52) U.S. Cl. CPC A61B 5/721; G06K 9/00342; A61B 5/1126; A61B 5/4809; A61B 5/024; A61B 5/0507; G01S 13/50; G01S 13/87; A61B 5/0205

(57) ABSTRACT

A sensor system includes a transmitter configured to transmit radio wave and a detector configured to detect a body motion when a change in amplitude of a received wave of a transmitted radio wave is detected and a lack of a frequency component indicating one or both of a heartbeat and a respiration in a frequency analysis result of the received wave is detected.

### CROSS-REFERENCE TO RELATED APPLICATION

This application is a continuation application of International Application PCT/JP2015/084558 filed on Dec. 9, 2015 and designated the U.S., the entire contents of which are incorporated herein by reference.

### FIELD

The embodiments discussed herein are related to a sensor system, a sensor information processing apparatus, a non-transitory computer-readable recording medium having stored therein a sensor information processing program, and a bed.

### BACKGROUND

A technology for measuring (which may be referred to as “detecting”) biological information such as heartbeat, respiration, and motion of a living body in a non-contact manner using, for example, a Doppler sensor has been studied and examined.

In addition, a technology for determining or estimating a state involving with sleep of a living body (which may be abbreviated as a “sleeping state”) based on biological information measured using a Doppler sensor has also been studied and examined.

### LIST OF RELATED ART DOCUMENTS

[Patent Document 1] Japanese National Publication of International Patent Application No. 2014-518728

[Patent Document 2] Japanese Laid-open Patent Publication No. 2011-50604

[Patent Document 3] Japanese Laid-open Patent Publication No. 2013-198654

[Patent Document 4] Japanese Laid-open Patent Publication No. 2010-99173

### SUMMARY

According to an aspect of the embodiments, a sensor system may include a transmitter configured to transmit radio wave, a receiver configured to receive a wave corresponding to the radio wave from the transmitter and a detector. The detector is configured to detect a body motion when a change in amplitude of the wave received by the receiver is detected and a lack of a frequency component indicating at least one of a heartbeat and a respiration in a frequency analysis result of the wave is detected.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a diagram illustrating a configuration example of a sensor system according to one embodiment.

FIG. 2 is a plan view schematically illustrating a configuration example of a multiuser compatible sensor attached bed according to one embodiment.

FIG. 3 is a side view schematically illustrating a configuration example of the multiuser compatible sensor attached bed illustrated in FIG. 2.

FIG. 4 is a plan view schematically illustrating another configuration example of a multiuser compatible sensor attached bed according to one embodiment.

FIG. 5 is a side view schematically illustrating a configuration example of the multiuser compatible sensor attached bed illustrated in FIG. 4.

FIG. 6 is a block diagram illustrating a configuration example of a Doppler sensor illustrated in FIGS. 1 to 5.

FIG. 7 is a block diagram illustrating a configuration example of an information processing apparatus illustrated in FIG. 1.

FIG. 8 is a flowchart illustrating an operation example (a first embodiment) of an information processing apparatus illustrated in FIGS. 1 and 7.

FIG. 9 is a flowchart illustrating an example of a body motion amount correction process illustrated in FIG. 8.

FIGS. 10A and 10B are diagrams illustrating an example of registration contents of a database (DB) illustrated in FIG. 8, where FIG. 10A is a diagram illustrating an example of registration contents before the body motion amount correction process and FIG. 10B is a diagram illustrating an example of registration contents after the body motion amount correction process.

FIG. 11 is a diagram illustrating an example of a change in time (a signal waveform) of a Doppler sensor value according to one embodiment.

FIG. 12 is a diagram illustrating an example of a frequency analysis result of the Doppler sensor value illustrated in FIG. 11.

FIG. 13 is a diagram illustrating an example of a signal waveform of a heartbeat component obtained based on the frequency analysis result illustrated in FIG. 12.

FIG. 14 is a diagram illustrating an example of a signal waveform of a respiration component obtained based on the frequency analysis result illustrated in FIG. 12.

FIG. 15 is a flowchart illustrating another operation example (a second embodiment) of the information processing apparatus illustrated in FIGS. 1 and 7.

FIG. 16 is a flowchart illustrating another operation example (the second embodiment) of the information processing apparatus illustrated in FIGS. 1 and 7.

FIG. 17 is a flowchart illustrating another operation example (a third embodiment) of the information processing apparatus illustrated in FIGS. 1 and 7.

FIG. 18 is a schematic diagram illustrating a comparative example of one embodiment.

### DESCRIPTION OF EMBODIMENTS

At the time of measuring the motion of each person by using a plurality of Doppler sensors, when a vibration or the like corresponding to one of the motions of a plurality of persons is transmitted to the other person, a change appears in the sensor value corresponding to the other person as if the other person has moved although the other person does not actually move.

For example, in a case in which a plurality of persons is sleeping on one bed and the motion (which may be referred to as a “body motion”) of each person is measured by a plurality of Doppler sensors, when someone rolls, the vibration is transmitted to the other sleeping person.

In this case, a change in amplitude appears in the sensor value of the Doppler sensor corresponding to the other person as if the other person has moved due to the influence of the vibration of the rolled person although the other person actually does not move.

For that reason, there is a possibility that the motion measurement accuracy of each person may be deteriorated. When the motion measurement accuracy of each person is deteriorated, the sleeping state estimation accuracy using the motion measurement result of each person may be deteriorated.

Embodiments will be described below with reference to the drawings. However, the embodiments described below are merely examples and it is not intended to exclude various modifications or applications of techniques not explicitly described below. In addition, various exemplary embodiments described below may be combined appropriately. In the drawings used in the following embodiments, the portions denoted by the same reference numerals indicate the same or similar portions unless otherwise specified.

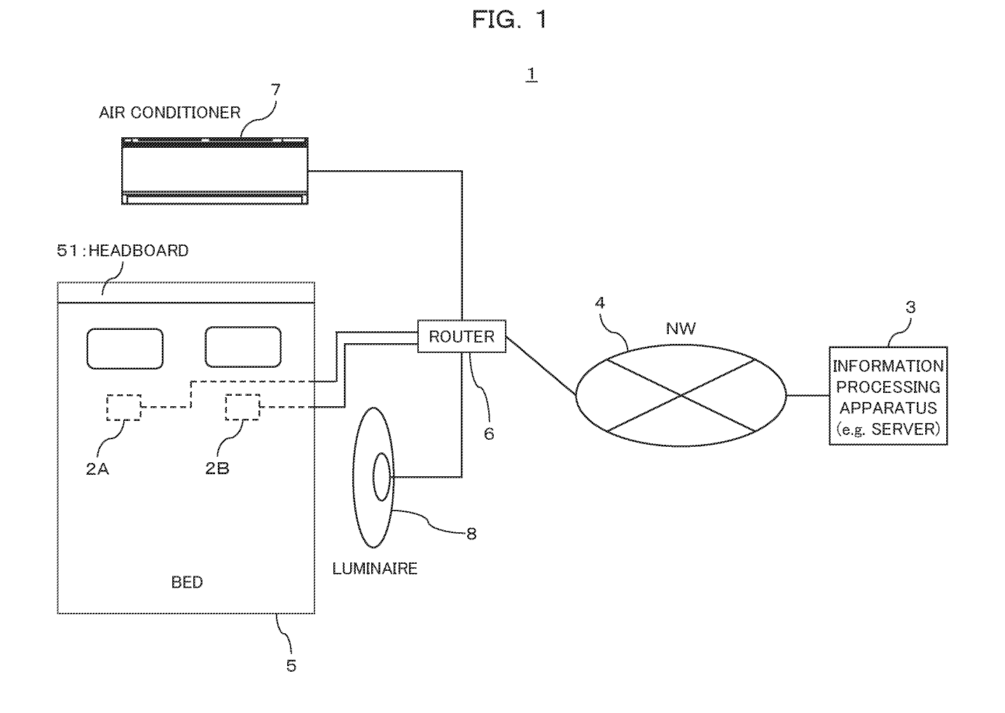

FIG. 1 is a block diagram illustrating a configuration example of a sensor system according to one embodiment. For example, a sensor system 1 illustrated in FIG. 1 may include a first sensor 2A, a second sensor 2B, and an information processing apparatus 3.

The sensors 2A and 2B may be connected to the information processing apparatus 3 via a network (NW) 4 to communicate with each other. For example, the sensors 2A and 2B may be connected to the network 4 via a router 6 which is an example of a communication device.

For example, the sensors 2A and 2B may be Doppler sensors and are able to detect a “motion” of a sensing target in a non-contact manner based on a change in wave reflected from the sensing target by irradiating a radio wave such as a microwave to the sensing target.

For example, when a distance between the sensor 2A (or 2B) and the sensing target changes, the reflected wave changes due to the Doppler effect. A change in reflected wave can be regarded as, for example, one or both of a change in amplitude of the reflected wave and a change in frequency of the reflected wave.

When the sensing target is a living body such as a human body, the distance between the sensor 2A (or 2B) and the sensing target changes according to the “motion” of the living body, so that biological information (referred to as “vital information”) can be sensed. In addition, “sensing” may be paraphrased as “detection” or “measurement”.

For example, the “motion” (which may be paraphrased as a “change in position”) of the living body is not limited to the physical motion during the activity of the living body but also includes the motion of the biological surface (for example, a skin) corresponding to a heartbeat or respiration at resting time such as in a sleeping state.

It may be understood that the motion of the biological surface is caused in accordance with the motion of the living organ. For example, the motion occurs in the skin in response to the beating of the heart. In addition, the motion of the skin occurs in response to the stretching of the lung accompanying respiration.

Since the reflection of the microwave irradiated by the sensor 2A (or 2B) changes due to the Doppler effect in response to the “motion” of the living body, it is possible to sense vital information indicating, for example, physical motion, heartbeat, and respiration based on the change.

In the following description, the “physical motion” of the human body is referred to as a “body motion” for convenience of description and is distinguished from the motion of the human body surface accompanying heartbeat, respiration, or the like. However, the “body motion” may include the physical motion of the human body and the motion of the human body surface accompanying heartbeat, respiration, or the like.

Based on the vital information sensed by the sensor 2A (or 2B), it is possible to detect, determine, or estimate the sleeping state of the living body in a non-contact manner, such as whether the living body is sleeping or awakened.

For example, the sensors 2A and 2B may be attached to a bed 5 which is an example of bedding provided in an indoor space such as a bedroom and may sense the vital information of the user of the bed 5 in a non-contact manner. In addition, the “user” may be referred to as an “observed person” or a “subject” by sensors 2A and 2B.

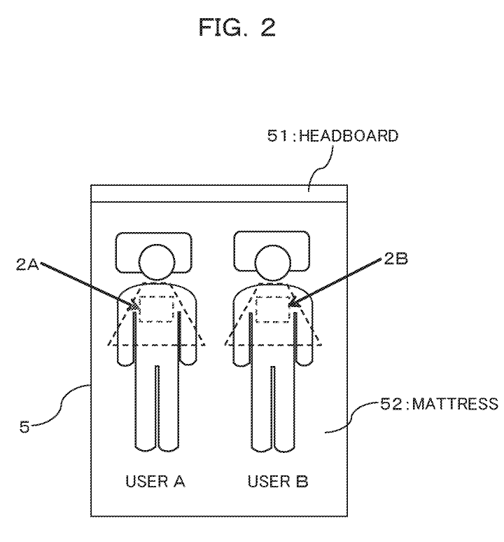

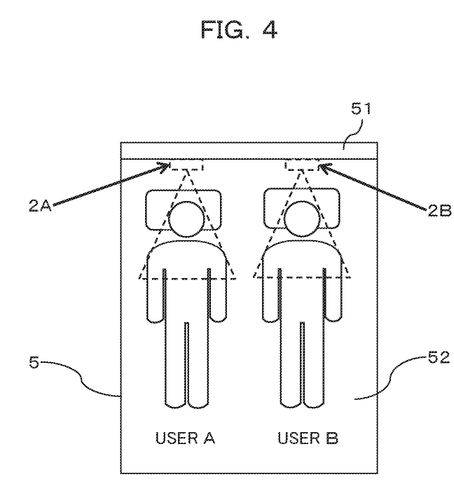

The bed 5 may be a bed in which two or more persons can sleep. For example, the bed 5 may be a bed having a width equal to or larger than a width (for example, 1400 mm) of a general double bed. In the following description, as a non-limiting example, it is assumed that the bed 5 is a double bed of a size that allows two users A and B to sleep side by side as illustrated in FIG. 2 or 4.

The sensors 2A and 2B may be attached to the bed 5 to respectively correspond to the users A and B.

For example, in the double bed 5, the first sensor 2A may be attached to the bed 5 so that a part or the entirety of a first sleeping region assumed to be occupied by one user A at bedtime is included in the sensing range.

The second sensor 2B may be attached to the double bed 5 so that a part or the entirety of a second sleeping region assumed to be occupied by the other user B at bedtime is included in the sensing range.

For example, the first and second sleeping regions may respectively correspond to left and right regions obtained by dividing the bed region of the double bed 5 in the width direction about the center line in the longitudinal direction.

As a non-limiting example, the first sensor 2A may be attached to a position in which the directivity of the transmission radio wave is formed with respect to the first sleeping region and the radio wave can be irradiated toward the first user A. The second sensor 2B may be attached to a position in which the directivity of the transmission radio wave is formed with respect to the second sleeping region and the radio wave can be irradiated toward the second user B.

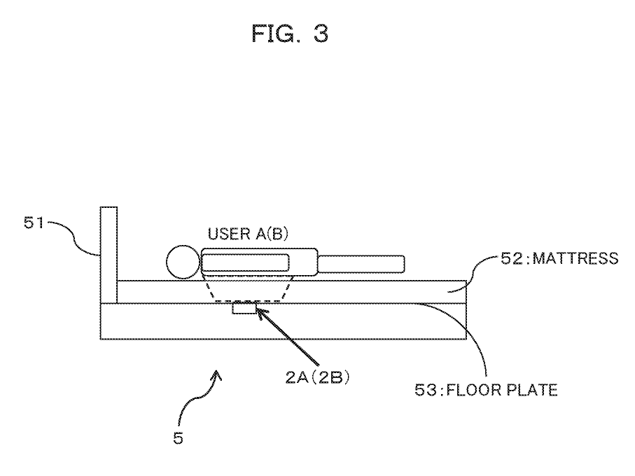

As an example of such an attachment position (which is sometimes referred to as a “sensor attachment position” for convenience of description), as schematically illustrated in FIGS. 2 and 3, a position in which the user A (or B) can be irradiated with radio waves from a rear side of a mattress 52 is exemplified.

For example, the first sensor 2A may be attached in a region corresponding to the sleeping region of the user A of a floor plate (which may be referred to as a “bottom plate”) 53 (see FIG. 3) of the bed 5 on which the mattress 52 is placed so that the directivity of the transmission radio wave is directed upward.

For example, the second sensor 2B may be attached in a region corresponding to the sleeping region of the user B of the floor plate 53 so that the directivity of the transmission radio wave is directed upward.

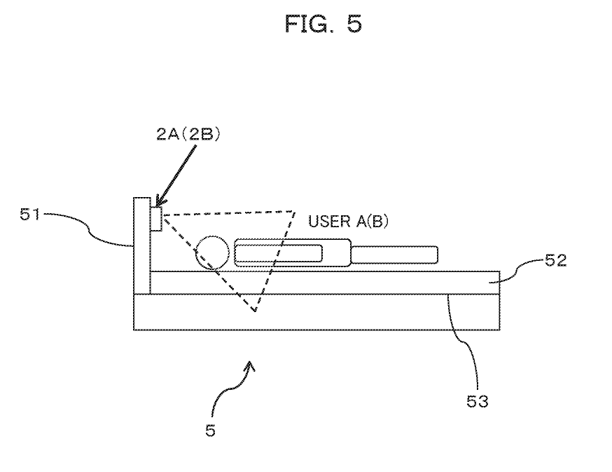

As another example of the sensor attachment position, as illustrated in FIGS. 4 and 5, a headboard 51 of the bed 5 is exemplified. The sensors 2A and 2B may be attached to the headboard 51 by embedding or external attaching.

For example, the sensors 2A and 2B may be attached depending on the height of the headboard 51 and may be attached to a position of several tens of centimeters (cm), that is, about 30 cm as a non-limiting example from the surface of the mattress 52 upward in the vertical direction.

As schematically illustrated in FIGS. 2 to 5, the sensing ranges of the sensors 2A and 2B may be respectively set to include the chests of the users A and B. By this setting, the heartbeat or respiration of the users A and B is easily measured. Further, the sensing ranges of the sensors 2A and 2B may be set not to overlap each other so that the radio wave interference can be avoided as much as possible.

For example, as will be described below, the sensing ranges of the sensors 2A and 2B may be respectively adjusted by controlling the transmission power of the radio waves.

As illustrated in FIGS. 2 and 3, in the embodiment in which the sensors 2A and 2B are attached to the floor plate 53 of the bed 5, it is easy to adjust the sensors so that a region including at least the chests of the users A and B is included in the sensing range and thus it is easy to measure the heartbeat or respiration of the users A and B.

Meanwhile, as illustrated in FIGS. 4 and 5, in the embodiment in which the sensors 2A and 2B are attached to the headboard 51 of the bed 5, for example, the sensors 2A and 2B hardly receive an influence of disturbance accompanied by a change in shape of the bed 5.

For example, even when the hardness of the mattress 52 of the bed 5 is changed or the shape of the reclinable bed 5 is changed, the influence on the sensing by the sensors 2A and 2B can be suppressed and thus a decrease in sensing accuracy can be suppressed.

In addition, the sensor attachment position illustrated in FIGS. 2 and 3 and the sensor attachment position illustrated in FIGS. 4 and 5 may be appropriately combined with each other. For example, one of the sensors 2A and 2B may be attached to the floor plate 53 of the bed 5 and the other thereof may be attached to the headboard 51 of the bed 5.

The bed 5 to which the sensors 2A and 2B are attached may be referred to as a “multiuser compatible sensor attached bed 5” for convenience of description.

Further, as schematically illustrated in FIG. 1, an air conditioner 7 or a luminaire 8 may be provided in an indoor space provided with the bed 5.

Similarly to the sensors 2A and 2B, the air conditioner 7 or the luminaire 8 may be connected to the router 6 and may be connected to the information processing apparatus 3 via the router 6 and the network 4 so that a communication therebetween is possible.

Operations of the air conditioner 7 or the luminaire 8 may be controlled from the information processing apparatus 3 by a communication via the router 6 and the network 4.

For example, the information processing apparatus 3 may remotely control the operation of the air conditioner 7 or the dimming of the luminaire 8 by using the sensing result of the sensors 2A and 2B. The control may be to control the environment in the indoor space (which may be referred to as an “indoor environment”) to a comfortable environment for the user.

For example, the control of the indoor environment using the information processing apparatus 3 may include the temperature control, the air volume control, or the wind direction control of the air conditioner 7 and the dimming control of the luminaire 8 which help the good sleep of the user. Such control may be referred to as “good sleep control” for convenience of description.

In addition, it is possible that the sensors 5A and 5B are not controlled by the information processing apparatus 3. In other words, the sensors 5A and 5B are enough to communicate with the information processing apparatus 3 in one direction and it is possible that the sensors 5A and 5B do not receive signals transmitted from the information processing apparatus 3.

A part or all of the sensors 2A and 2B, the air conditioner 7, and the luminaire 8 may be connected to the router 6 in a wired or wireless manner. The air conditioner 7 or the luminaire 8 may be either for home use or business use. The air conditioner 7 or the luminaire 8 for home is an example of a so-called “home appliance” and the “home appliance” capable of communicating with the network 4 may be referred to as an “information home appliance”.

For example, the network 4 may correspond to a Wide Area Network (WAN), a Local Area Network (LAN), or an internet. The network 4 may include a wireless access network. For example, the router 6 may be connected to the wireless access network via a wireless interface and may communicate with the information processing apparatus 3.

As described above, the information processing apparatus 3 can receive (which may be referred to as “acquire”) the sensor information of the sensors 2A and 2B via the network 4. Thus, the information processing apparatus 3 may be referred to as the sensor information processing apparatus 3.

Based on the received sensor information, the information processing apparatus 3 can determine (which may be referred to “estimate”) states involving with the body motion, the heartbeat, or the respiration of the users A and B. Based on the estimation result, the information processing apparatus 3 may control the indoor environment as described above.

For example, the information processing apparatus 3 may be configured by using one or a plurality of servers. In other words, one server may correspond to the information processing apparatus 3 and a server system including a plurality of servers may correspond to the information processing apparatus 3. The server may correspond to, for example, a cloud server provided in a cloud data center.

(Configuration Examples of Sensors 2A and 2B)

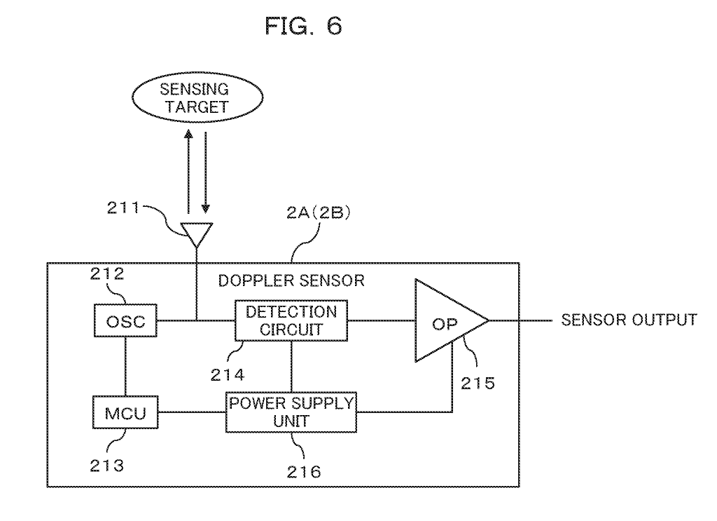

Next, configuration examples of the sensors 2A and 2B will be described with reference to FIG. 6. Additionally, the configuration examples illustrated in FIG. 6 may be common to the sensors 2A and 2B. For that reason, when the sensors 2A and 2B are not distinguished structurally, the sensors 2A and 2B may be abbreviated as a “sensor 2” in some cases.

The sensor 2 illustrated in FIG. 6 is a Doppler sensor. The Doppler sensor 2 may be referred to as a “microwave sensor 2” or an “RF sensor 2”. “RF” is an abbreviation for “Radio Frequency”.

For example, the Doppler sensor 2 generates a beat signal by phase-detecting the transmission radio wave and the reflected wave of the transmitted radio wave. For that reason, as illustrated in FIG. 6, the Doppler sensor 2 may include, for example, an antenna 211, a local oscillator (Oscillator, OSC) 212, a Micro Control Unit (MCU) 213, a detection circuit 214, an operational amplifier (OP) 215, and a power supply unit 216.

The antenna 211 transmits a radio wave having an oscillation frequency generated by the OSC 212 and receives the reflected wave of the transmission radio wave. Additionally, in the example of FIG. 6, the antenna 211 is commonly used for transmission and reception, but may be individually used for each of the transmission and reception.

For example, the OSC 212 is oscillated by the control of the MCU 213 and outputs a signal (which may be referred to as a “local signal” for convenience of description) having a predetermined frequency. The local signal is transmitted from the antenna 211 as the transmission radio wave and is input to the detection circuit 214.

For example, the oscillation frequency of the OSC 212 may be a frequency in a microwave band. The microwave band may be, for example, a 2.4 GHz band or a 24 GHz band. These frequency bands are examples of frequency bands that are allowed to be used indoors under the Radio Law of Japan. A frequency band that is not regulated by the Radio Law may be used as the transmission radio wave of the Doppler sensor 2.

For example, the MCU 213 controls the oscillation operation of the OSC 212.

The detection circuit 214 outputs a beat signal by phase-detecting the reflected wave received by the antenna 211 and the local signal (in other words, the transmission radio wave) from the OSC 212. In addition, the detection circuit 214 may be replaced by a mixer that mixes the transmission radio wave and the reflected wave. It may be understood that the mixing by the mixer is equal to the phase-detecting.

Here, the beat signal obtained by the detection circuit 214 has a change in amplitude and frequency occurring in response to the “motion” of the user A or B reflecting the transmission radio wave due to the Doppler effect.

For example, the frequency and the amplitude value of the beat signal tend to increase as the change amount of the “motion” (in other words, the relative speed to the Doppler sensor 2) increases.

In other words, the beat signal includes information indicating the “motion” of the sensing target (for example, the user A or B) reflecting the transmission radio wave. As described above, examples of the “motion” of the sensing target include the body motion which is a physical motion of the user and the motion of the human body surface (in other words, a skin) accompanied by the heartbeat or respiration.

Since a distance between the user and the Doppler sensor 2 changes due to a change of the human body surface in accordance with the heartbeat or respiration, the waveform of the beat signal changes in response to a change in distance. Thus, it is possible to detect the heart rate or the respiratory rate of the user as well as the body motion of the user based on a change in waveform of the beat signal.

For example, since the body motion of the user has a tendency that the amplitude value of the beat signal largely changes compared to the motion of the human body surface in response to the heartbeat or respiration of the user, the body motion can be detected based on a change in amplitude value.

On the contrary, since the motion of the human body surface in response to the heartbeat or respiration of the user easily appears as a change in frequency compared to a change in amplitude value in the beat signal, the motion of the human body surface can be detected based on a change in frequency.

The operational amplifier 215 amplifies the beat signal output from the detection circuit 214. The amplified beat signal may be transmitted to the information processing apparatus 3 as the sensor information.

The power supply unit 216 supplies driving power to, for example, the MCU 213, the detection circuit 214, and the operational amplifier 215.

In addition, the oscillation frequency and the output signal strength of the OSC 212 may be the same or different in the Doppler sensor 2A and the Doppler sensor 2B. In other words, the frequency and the power of the radio wave transmitted by the Doppler sensors 2A and 2B may be the same or different.

It may be said that the power of the transmission radio wave is the “transmission radio wave strength” or the “transmission power”. Since the reachable space range of the radio wave increases as the transmission power increases, the sensing range can be expanded. The transmission power of the Doppler sensors 2A and 2B may be individually set or adjusted in response to the distance between the sensor attachment position and the sensing target.

(Configuration Example of Information Processing Apparatus 3)

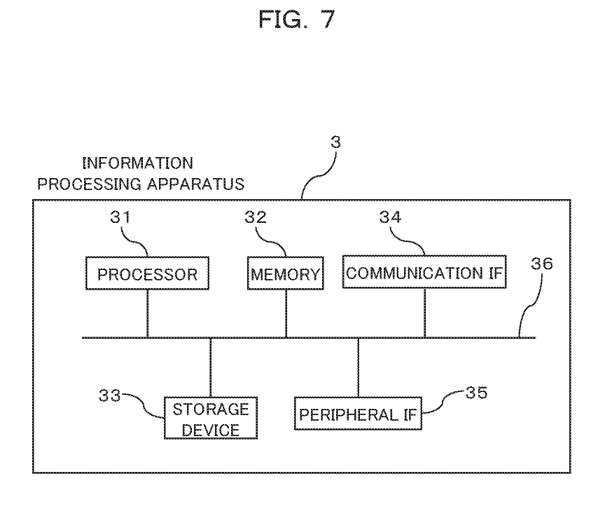

Next, a configuration example of the information processing apparatus 3 illustrated in FIG. 1 will be described with reference to FIG. 7. As illustrated in FIG. 7, the information processing apparatus 3 may include, for example, a processor 31, a memory 32, a storage device 33, a communication interface (IF) 34, and a peripheral IF 35.

The processor 31, the memory 32, the storage device 33, the communication IF 34, and the peripheral IF 35 may be connected to each other to communicate with each other via, for example, a communication bus 36.

The processor 31 is an example of a processing unit and controls, for example, the entire operation of the information processing apparatus 3. The control may include communication control via the network 4. The control may include remote control for one or both of the air conditioner 7 and the luminaire 8 via the network 4.

For example, the processor 31 may determine the sleeping states of the users A and B based on the sensor information of the Doppler sensors 2A and 2B received by the communication IF 34 and may generate a control signal for controlling the operation of the air conditioner 7 or the luminaire 8 in response to the determination result. The control signal may be transmitted to the air conditioner 7 or the luminaire 8 via, for example, the communication IF 34.

The processor 31 is an example of an arithmetic processing apparatus having arithmetic capability. The arithmetic processing apparatus may be referred to as an arithmetic device or an arithmetic circuit. For example, a CPU may be applied to the processor 31 which is an example of the arithmetic processing apparatus. The “CPU” is an abbreviation for “Central Processing Unit”.

Instead of the CPU, for example, an Integrated Circuit (IC) such as a Micro Processing Unit (MPU) or a Digital Signal Processor (DSP) may be used in the processor 31. Additionally, the “arithmetic processing apparatus” may be referred to as a “computer”.

The memory 32 is an example of a storage medium and may be a Random Access Memory (RAM) or a flash memory. A program or data read by the processor 31 and used for an operation may be stored in the memory 32. The “program” may be referred to as “software” or “application”.

The storage device 33 may store various data or program. As the storage device 33, a hard disk drive (HDD), a solid state drive (SSD), a flash memory, or the like may be used.

The data stored in the storage device 33 may include, for example, the sensor information of the Doppler sensors 2A and 2B received by the communication IF 34, the vital information obtained based on the sensor information, the sleeping state determination result estimated based on the vital information, and the like.

The data stored in the storage device 33 may be appropriately collected into a database (DB). The DB data may be referred to as “cloud data” or “big data”. Additionally, the storage device 33 and the memory 32 may be generally referred to as a “storage unit”.

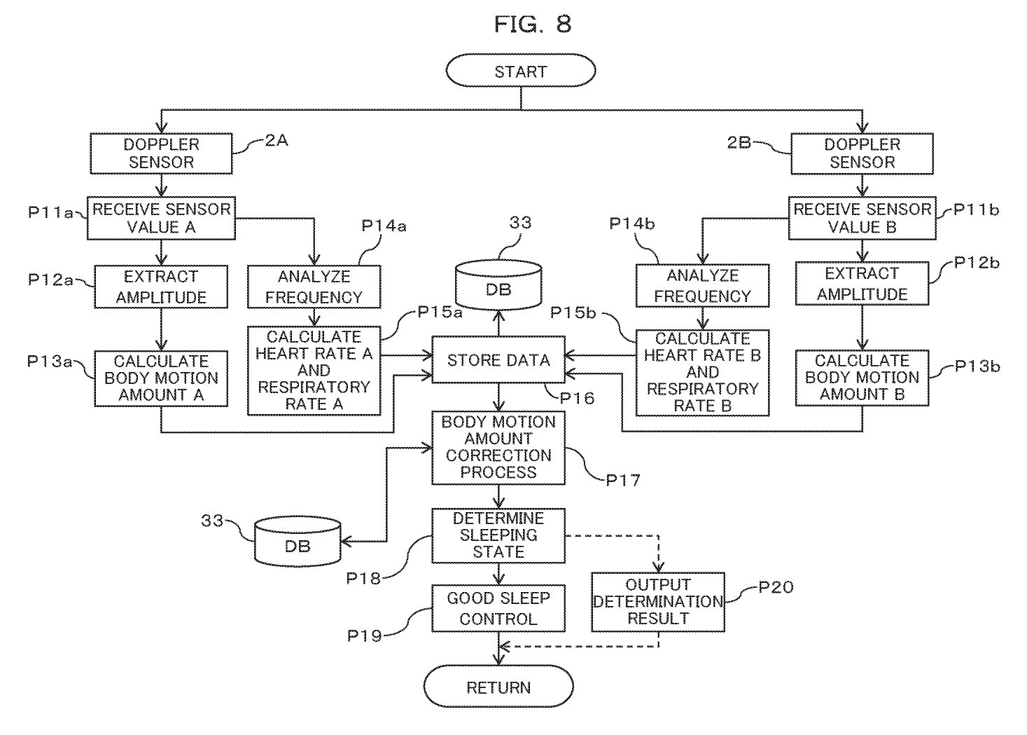

The program stored in the storage device 33 may include a program that executes a process (which may be referred to as a “sensor information process”) which will be described later in FIG. 8 or 9 and FIGS. 15 to 17. For convenience of description, the program may be referred to as a “sensor information processing program”. A part or the entirety of a program code constituting the program may be stored in the storage unit or may be described as a part of an operating system (OS).

The program or data may be provided while being stored in a computer readable recording medium. As an example of the recording medium, a flexible disk, a CD-ROM, a CD-R, a CD-RW, a MO, a DVD, a Blu-Ray Disc, a portable hard disk, and the like can be exemplified. Further, a semiconductor memory such as a Universal Serial Bus (USB) memory is also an example of the recording medium.

Alternatively, the program or data may be provided (downloaded) from the server or the like to the information processing apparatus 3 via the network 4. For example, the program or data may be provided to the information processing apparatus 3 via the communication IF 34. Further, the program or data may be input to the information processing apparatus 3 from an input unit which will be described later and is connected to the peripheral IF 35.

The communication IF 34 is connected to, for example, the network 4 so that a communication is possible via the network 4.

Focusing on the reception process, the communication IF 34 is an example of a receiver (which may be referred to as an “acquiring unit”) which receives information transmitted from the sensors 2A and 2B to the information processing apparatus 3.

Meanwhile, focusing on the transmission process, the communication IF 34 is, for example, an example of a transmitter which transmits, for example, a control signal which is generated by the processor 31 to the air conditioner 7 or the luminaire 8. For example, Ethernet (registered trademark) cards may be applied to the communication IF 34.

The peripheral IF 35 is, for example, an interface for connecting a peripheral device to the information processing apparatus 3.

The peripheral device may include an input unit which inputs information to the information processing apparatus 3 or an output unit which outputs information generated by the information processing apparatus 3.

The input unit may include a keyboard, a mouse, a touch panel, or the like. The output unit may include a display, a printer, or the like.

(Operation Example)

Hereinafter, an operation example of the sensor system 1 will be described.

In the following operation example, an example of estimating the sleeping state of a plurality of (for example, two) users A and B respectively by the information processing apparatus 3 based on the sensor information obtained by the sensors 2A and 2B in a non-contact manner will be described.

Further, in the following description, there is a case in which the sensor information which is the sensing result of the Doppler sensors 2A and 2B is referred to as a “detection value” or a “sensor value”. Further, there is a case in which the sensor value of the Doppler sensor 2A corresponding to the user A is referred to as a “Doppler sensor value A” or a “sensor value A” for convenience of description. Similarly, there is a case in which the sensor value of the Doppler sensor 2B corresponding to the user B is referred to as a “Doppler sensor value B” or a “sensor value B” for convenience of description.

For example, when two users A and B sleep on one bed 5 side by side, the body motion, the heartbeat, or the respiration of each user can be measured by providing the Doppler sensors 2A and 2B to correspond to their sleeping positions (in other words, the sleeping regions) as illustrated in FIGS. 2 to 5.

However, when the physical motion (the body motion) in which someone (for example, the user A) rolls occurs as a plurality of persons sleep on one bed 5, a vibration corresponding to the body motion is transmitted to, for example, the mattress 52 or the futon (comforter, duvet) of the bed 5 so that the other user B may also move.

In this case, a change in amplitude occurs in the sensor value B corresponding to the other user B as if the body motion of the user B occurs although the user B actually does not move.

For that reason, a false motion detection for the user B occurs although the user B actually does not move. Accordingly, the body motion detection accuracy of the user B is deteriorated and thus the sleeping state estimation accuracy is also deteriorated.

In contrast, when the body motion of the user B occurs due to the rolling motion, a vibration is transmitted to the user A and thus a change in amplitude occurs in the sensor value A as if the body motion of the user A occurs.

For that reason, the body motion detection accuracy of the user A is deteriorated and thus the sleeping state detection accuracy of the user A is detonated.

In this way, in a case in which the plurality of users A and B sleep on one bed 5, even when the sensors 2A and 2B are provided to respectively correspond to the users A and B, the body motion detection accuracy for the other person is deteriorated when the body motion occurs in any one of the users A and B due to the rolling motion.

Even when the radio wave frequencies of the sensors 2 corresponding to the plurality of users are different from each other, a change in amplitude caused by the vibration of the user due to the body motion such as rolling motion occurs in the sensor value of the sensor 2 corresponding to the other user and thus the body motion detection accuracy can be deteriorated.

Since information indicating whether the body motion occurs while sleeping is information used to estimate the quality (for example, the depth) of the sleeping, it is desirable to suppress a false detection as if someone who is not moving has moved as much as possible.

Here, the user with the body motion such as rolling motion and the user without the body motion can be distinguished from each other by, for example, the data of the specific frequency component in the frequency analysis result for the Doppler sensor value.

Fast Fourier Transform (FFT) or Discrete Fourier Transform (DFT) may be used for frequency analysis. The specific frequency component is, for example, a frequency component indicating one or both of the heartbeat and the respiration of the human body. In addition, the frequency component indicating the heartbeat may be briefly referred to as a “heartbeat component” and the frequency component indicating the respiration may be briefly referred to as a “respiration component”.

There is a tendency that the heartbeat component has a peak in the frequency range higher than that of the respiration component in the frequency analysis result. For example, there is a tendency that the heartbeat component of the human body has a peak frequency in the frequency range of about 0.7 Hz to 3 Hz and the respiration component of the human body has a peak frequency in the frequency range of about 0.1 Hz to 0.3 Hz.

When the user largely moves while rolling on the bed 5, a waveform disturbance corresponding to the body motion occurs in the Doppler sensor value and thus the data corresponding to one or both of the heartbeat component and the respiration component lack in the frequency analysis result (or it becomes difficult to discriminate, hereinafter the same).

On the contrary, although a temporary waveform disturbance occurs in the sensor value corresponding to the other user without the body motion while sleeping on the same bed 5, the heartbeat component and the respiration component easily exist in the frequency analysis result to be discriminated.

Thus, it is possible to determine or estimate which user corresponding to the Doppler sensor 2 has the body motion such as rolling motion based on the lack of the data corresponding to one or both of the heartbeat component and the respiration component in the frequency analysis result of the sensor value indicating the detection of the body motion.

For example, the sensor value in which the lack of one or both of the heartbeat component and the respiration component is detected in the frequency analysis result among the plurality of sensor values indicating a change in amplitude in the body motion detection indicates a state where the body motion such as rolling motion occurs in the user corresponding to the sensor value. Thus, the body motion amount obtained from the sensor value may be processed as a valid data.

Meanwhile, the sensor value in which the lack of the heartbeat component and the respiration component is not detected among the plurality of sensor values indicating a change in amplitude of the body motion detection indicates a state where no body motion occurs in the user corresponding to the sensor value. Thus, the body motion amount obtained from the sensor value may be corrected without processing it as a valid data.

With the above-described process, it is possible to improve the body motion detection accuracy for each user and to further improve the sleeping state estimation accuracy.

In addition, the body motion amount may be corrected such that, for example, the body motion amount is set to an invalid value, for example, 0. In other words, the correction of the body motion amount may be regarded as a process of masking the body motion amount or a process of not allowing the body motion detection as the normal (or valid) body motion detection.

In other words, the process of not allowing the body motion detection as a valid body motion detection may be regarded as a process of setting the body motion detection as an abnormal (or invalid) body motion detection (or a false body motion detection) or a process of ignoring the body motion detection.

### First Embodiment

Hereinafter, a first embodiment of a process by the information processing apparatus 3 will be described with reference to FIGS. 8 to 14.

Additionally, in the following description, there is a case in which the body motion amount, the heart rate, and the respiratory rate calculated for the user A are respectively expressed as the “body motion amount A”, the “heart rate A”, and the “respiratory rate A” for convenience of description. Similarly, there is a case in which the body motion amount, the heart rate, and the respiratory rate calculated for the user B are respectively expressed as the “body motion amount B”, the “heart rate B”, and the “respiratory rate B” for convenience of description.

As illustrated in FIG. 8, the information processing apparatus 3 receives the Doppler sensor values A and B transmitted from the Doppler sensors 2A and 2B to the information processing apparatus 3 (Processes P11a and P11b). For example, the Doppler sensor values A and B are received by the communication IF 34 of the information processing apparatus 3 and are input to the processor 31 of the information processing apparatus 3.

For example, the processor 31 may extract amplitude components of the Doppler sensor values A and B (Processes P12a and P12b) and may calculate the body motion amount A of the user A and the body motion amount B of the user B based on the extracted amplitude components (Processes P13a and P13b).

For example, the processor 31 may determine the amplitude component exceeding a determination threshold value as the “body motion detection” by comparing the amplitude component and the determination threshold value with each other and may calculate the amplitude component determined as the “body motion detection” by the integration per unit time. Alternatively, the processor 31 may calculate the “body motion amount” as the numerical data indicating whether the “body motion detection” exists or not. For example, the existence of the “body motion detection” may be indicated by “1” and the non-existence of the “body motion detection” may be indicated by “0”.

Along with the body motion amount calculation process, the processor 31 may analyze the frequencies of the sensor values A and B (Processes P14a and P14b) and may calculate the heart rates and the respiratory rates for the users A and B based on the frequency analysis result (Processes P15a and P15b).

For example, the sensor values A and B are respectively converted from time domain signals to frequency domain signals (which are referred to as “frequency signals” for convenience of description) by FFT processing.

The processor 31 may detect the frequency component (which may be referred to as a “FFT peak frequency” for convenience of description) indicating a relatively large change from the frequency signals of the sensor values A and B. The FFT peak frequency of the Doppler sensor value is an example of the frequency component indicating a specific change in response to the heartbeat or respiration.

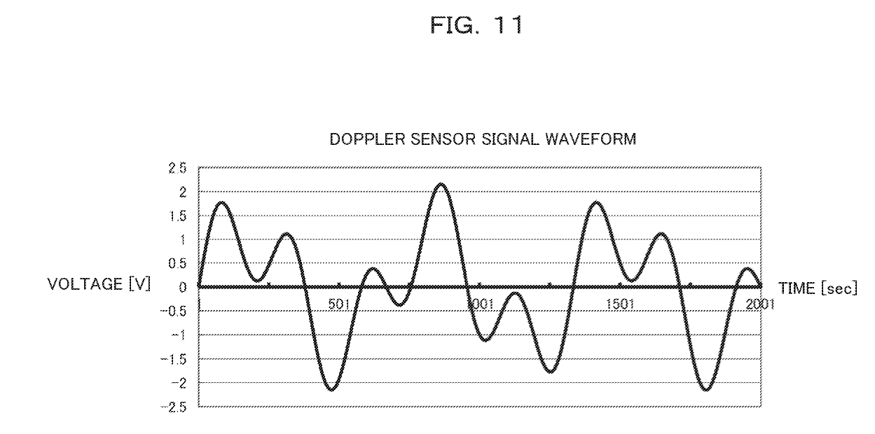

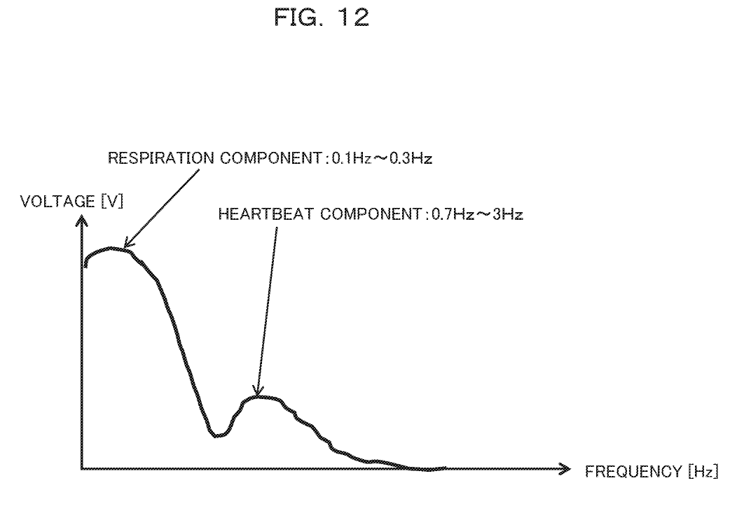

FIG. 11 illustrates an example of a change in Doppler sensor value with time and FIG. 12 illustrates an example of a FFT result of the Doppler sensor value illustrated in FIG. 11.

As illustrated in FIG. 12, there is a tendency that the heartbeat component of the human body has a peak frequency in the frequency range of about 0.7 Hz to 3 Hz in the FFT result of the Doppler sensor value as described above. Further, there is a tendency that the respiration component of the human body has a peak frequency in the frequency range of about 0.1 Hz to 0.3 Hz.

Thus, the processor 31 can separate the original signal waveform of the Doppler sensor value illustrated in FIG. 11 into the signal waveform corresponding to the respiration component and the signal waveform corresponding to the heartbeat component based on the peak frequency corresponding to the heartbeat component and the peak frequency corresponding to the respiration component.

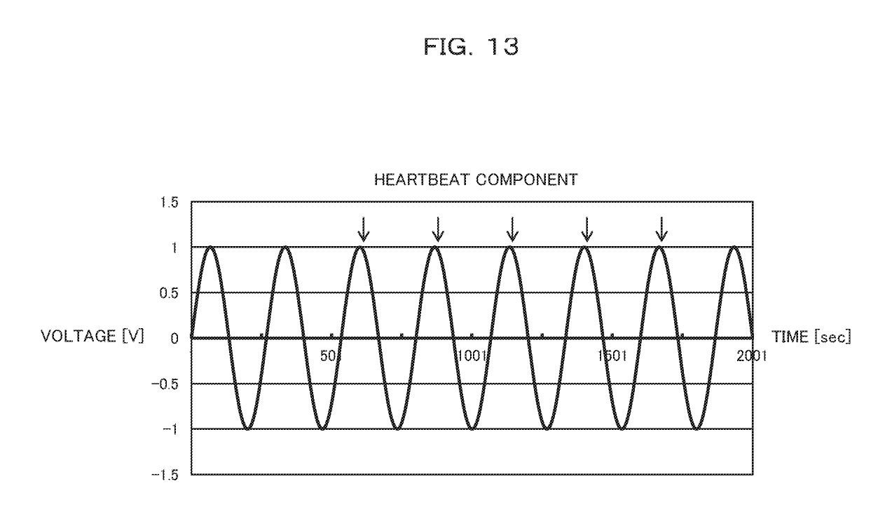

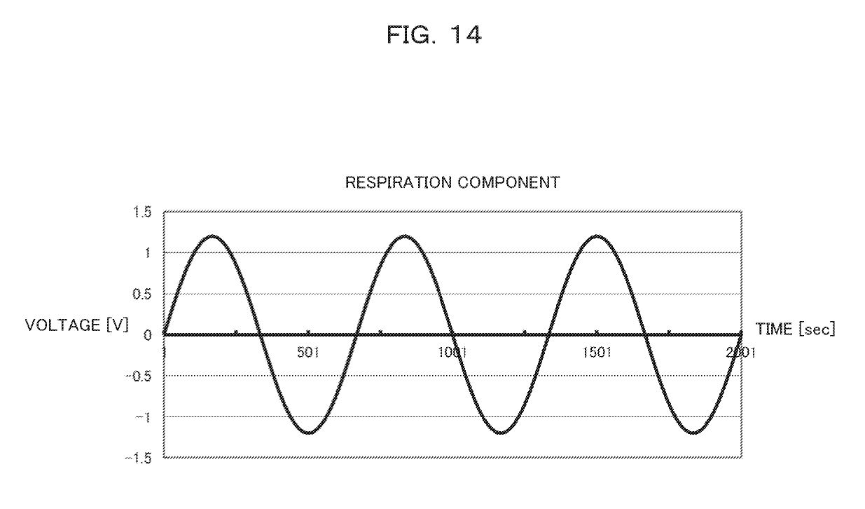

FIG. 13 illustrates an example of the signal waveform corresponding to the heartbeat component and FIG. 14 illustrates an example of the signal waveform corresponding to the respiration component.

The processor 31 may appropriately apply low pass filtering (LPF) processing for removing noise components of the separated signal waveforms.

The processor 31 can calculate the heart rate and the respiratory rate from the obtained signal waveform. For example, for the heart rate, the processor 31 may identify a feature point (for example, an amplitude peak) of the signal waveform corresponding to the heartbeat component and obtain the time interval (for example, “second”) of the feature points.

For example, the processor 31 may calculate the heart rate per minute by dividing 1 minute (=60 seconds) by the obtained time interval. The respiratory rate can be also calculated similarly by the processor 31.

Returning to FIG. 8, the processor 31 may store the body motion amounts, the heart rates, and the respiratory rates of the users A and B obtained by Processes P13a and P13b and Processes P15a and P15b in, for example, the storage device 33 (see FIG. 7) and collect them into the database (DB) (Process P16). In addition, there is a case in which the DB stored in the storage device 33 is referred to as a “DB 33” for convenience of description.

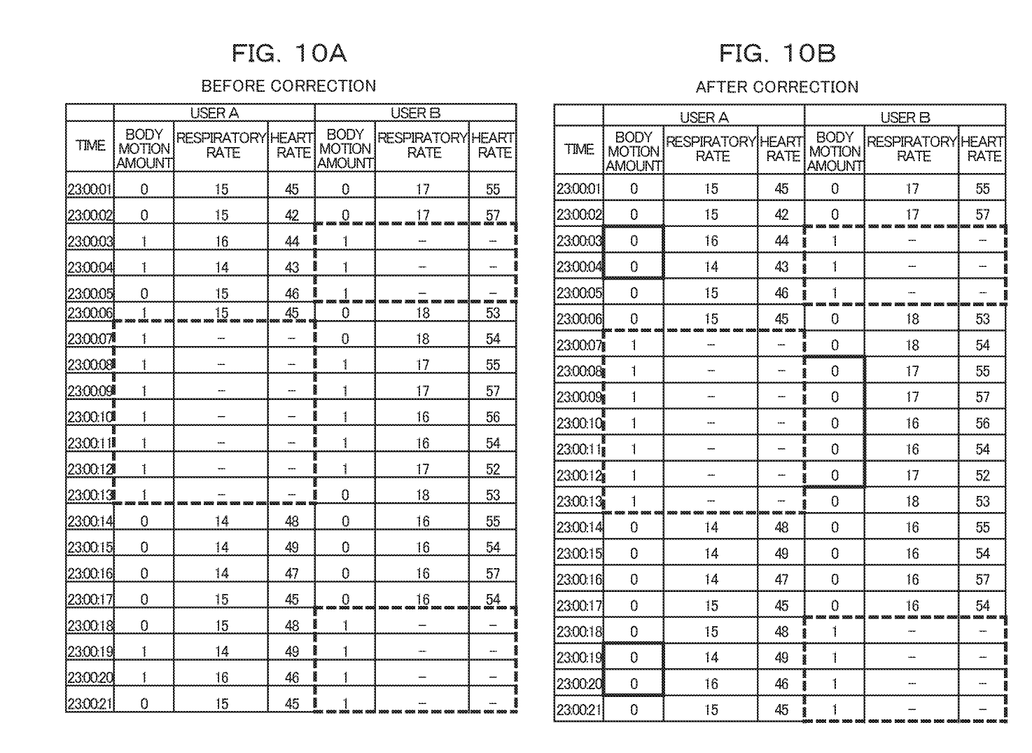

FIGS. 10A and 10B illustrate an example of the registration contents of the DB 33. In addition, FIG. 10A illustrates an example of the registration contents before the correction by the body motion amount correction process to be described later (Process P17 in FIG. 8) and FIG. 10B illustrates an example of the registration contents after the correction by the correction process.

As illustrated in FIGS. 10A and 10B, the DB 33 may register the body motion amount, the heart rate, and the respiratory rate for each of the users A and B every predetermined time (for example, 1 second). In addition, a portion surrounded by a dotted line in FIG. 10A indicates the lack of the respiratory rate and the heart rate of the user A or B due to the body motion such as rolling motion.

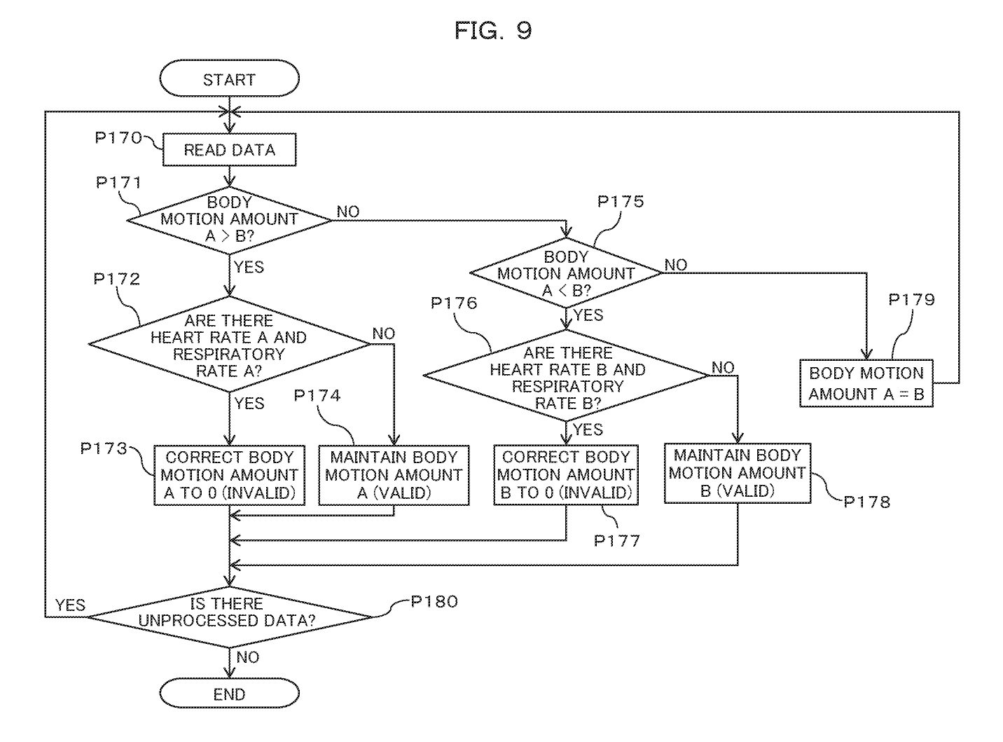

As illustrated in FIG. 8, the processor 31 may perform the body motion amount correction process based on the registration contents of the DB 33 illustrated in FIG. 10A (Process P17). FIG. 9 illustrates an example of the body motion amount correction process.

As illustrated in FIG. 9, the processor 31 may read data with reference to the DB 33 (Process P170) and determine whether the relationship of the body motion amount A>the body motion amount B is established by comparing, for example, the body motion amounts A and B of the users A and B at the same time (Process P171).

When the relationship of the body motion amount A>the body motion amount B is established as the determination result (YES in Process P171), the processor 31 may further determine whether the heart rate A and the respiratory rate A at the time of comparing the body motion amounts A and B are registered in the DB 33 (Process P172).

When one or both of the heart rate A and the respiratory rate A are not registered and lack as the determination result (NO in Process P172), the processor 31 may maintain the body motion amount A as the valid data by determining that the body motion such as rolling motion occurs in the user A (Process P174).

In other words, the processor 31 may process the body motion detection based on a change in amplitude of the Doppler sensor value A as the valid body motion detection. The process may be regarded as a process of detecting the body motion based on a change in amplitude of the Doppler sensor value A.

Meanwhile, when the heart rate A and the respiratory rate A are registered (YES in Process P172), the processor 31 may correct the body motion amount A to the invalid data (for example, 0) by determining that the body motion amount A is the data erroneously detected by the influence of the body motion of the other user B (Process P173).

For example, in the examples of FIGS. 10A and 10B, the body motion amount A (“1” in FIG. 10A) surrounded by the solid line for the user A in FIG. 10B is corrected to “0”.

In other words, it is possible that the processor 31 does not process the body motion detection based on a change in amplitude of the Doppler sensor value A as the valid body motion detection. The process may be regarded as a process of ignoring or not performing the body motion detection based on a change in amplitude of the Doppler sensor value A.

Further, in Process P171 for comparison, when the relationship of the body motion amount A>the body motion amount B is not established (NO in Process P171), the processor 31 may determine whether the relationship of the body motion amount A<the body motion amount B is established (Process P175).

When the relationship of the body motion amount A<the body motion amount B is established as the determination result (YES in Process P175), the processor 31 may further determine whether the heart rate B and the respiratory rate B at the time of comparing the body motion amounts A and B are registered in the DB 33 (Process P176).

When one or both of the heart rate B and the respiratory rate B are not registered and lack as the determination result (NO in Process P176), the processor 31 may maintain the body motion amount B as the valid data by determining that the body motion such as rolling motion occurs in the user B (Process P178).

In other words, the processor 31 may process the body motion detection based on a change in amplitude of the Doppler sensor value B as the valid body motion detection. The process may be regarded as a process of detecting the body motion based on a change in amplitude of the Doppler sensor value B.

Meanwhile, when the heart rate B and the respiratory rate B are registered (YES in Process P176), the processor 31 may correct the body motion amount B to the invalid data (for example, 0) by determining that the body motion amount B is the data erroneously detected by the influence of the body motion of the other user A (Process P177).

For example, in the examples of FIGS. 10A and 10B, the body motion amount B (“1” in FIG. 10A) surrounded by the solid line for the user B in FIG. 10B is corrected to “0”.

In other words, it is possible that the processor 31 does not process the body motion detection based on a change in amplitude of the Doppler sensor value B as the valid body motion detection. The process may be regarded as a process of ignoring or not performing the body motion detection based on a change in amplitude of the Doppler sensor value B.

Additionally, in Process P175 for comparison, when the relationship of the body motion amount A<the body motion amount B is not established (NO in Process P175), the processor 31 may determine the relationship of the body motion amount A=the body motion amount B is established (Process P179), may process (maintain) the body motion amounts A and B as the valid data, and may return the routine to Process P170.

The processor 31 may repeat the process after Process P170 (YES in Process P180) until no unprocessed data is left in the DB 33 (until NO in Process P180).

When no unprocessed data is left (NO in Process P180), the processor 31 may determine the sleeping states of the users A and B, for example, based on the data of the body motion amounts, the heart rates, and the respiratory rates of the users A and B registered in the DB 33 as illustrated in FIG. 8 (Process P18).

For example, the processor 31 may compare the “body motion amount” obtained over a certain unit time with a threshold value and may determine the time when the “body motion amount” is equal to or larger than the threshold value as the time when the user A or B is “awakened”.

In other words, the processor 31 may determine that the user A or B is “sleeping” at the time except for the time determined as “awakened”. The processor 31 may determine that the user A or B is “sleeping” when the time determined as “sleeping” is continued over a threshold time or more such as several minutes.

Further, the processor 31 may determine the depth of the sleep, for example, whether the sleep is the “REM sleep” or the “Non-REM sleep” based on the heart rate and the respiratory rate at the time determined that the user A or B is “sleeping”.

For example, the sleep cycle (or stage) of the user can be classified into Stages 1 to 5. Stage 1 is referred to as a “sleep onset stage”, Stage 2 is referred to as a “light sleep stage”, Stage 3 is referred to as a “moderate sleep stage” and Stage 4 is referred to as a “deep sleep stage”. Stages 1 to 4 are referred to as “non-REM sleep” and Stage 5 is referred to as “REM sleep”.

The processor 31 can determine, for example, the “Non-REM sleep” of Stages 3 and 4 and the “REM sleep” of Stage 5 based on the heart rate, the respiratory rate, and the body motion amount of the user.

For example, in the “REM sleep”, there is a tendency that the heart rate increases and irregularly changes and the respiratory rate increases. Meanwhile, this is a level which may be determined that the body motion amount does not exist or substantially does not exist.

On the contrary, in the “Non-REM sleep”, there is a tendency that the heart rate decreases and the respiratory rate decreases to be stabilized. Meanwhile, this is a level which may be determined that the body motion amount does not exist or substantially does not exist.

Thus, the processor 31 can determine whether the sleep of the users A and B is the “REM sleep” or the “Non-REM sleep” based on the tendency of a change in the heart rate and the respiratory rate of each of the “REM sleep” and the “Non-REM sleep”.

The processor 31 may control the indoor environment provided with the bed 5 based on the sleeping state determination result. For example, since the processor 31 can estimate the respective sleeping states of a plurality of persons sleeping in a certain indoor space, the indoor environment can be adapted to each person.

For example, the processor 31 may perform “good sleep control” such as temperature control, air volume control, wind direction control, and dimming control which help the good sleep of each of the users A and B by controlling the operation of the air conditioner 7 or the luminaire 8 based on the sleeping state determination result (Process P19).

For example, the sleeping state determination result can be used in the information for controlling the wind direction of the air conditioner 7 or the dimming of the luminaire 8 for each person.

Additionally, the sleeping state determination result of the users A and B may be appropriately output to an external device (not illustrated) as a report or the like (Process P20). The external device may be a display or a printer.

Here, since the body motion amount used to determine the sleeping state as described above is corrected as described in FIG. 9, it is possible to reduce the possibility of the false detection that a person who is not moving has moved. In other words, it is possible to improve the body motion detection accuracy of each of the users A and B and to improve the sleeping state determination accuracy of each of the users A and B.

Additionally, the above-described example is an example in which two users A and B sleep on one bed 5, but even when three or more users sleep on one bed 5, this case can be handled by providing the Doppler sensor 2 in the bed 5 to correspond to each user.

For example, when one of the sensor values of three or more Doppler sensors 2 has a change in amplitude in the body motion detection, it may be determined that the body motion such as rolling motion occurs in the user corresponding to the sensor value in which the heartbeat component and the respiration component lack in the frequency analysis result of each sensor value. Then, the body motion detected in the user corresponding to the other sensor value may be set to be invalid by determining that the body motion is caused by the body motion of the rolling user.

### Second Embodiment

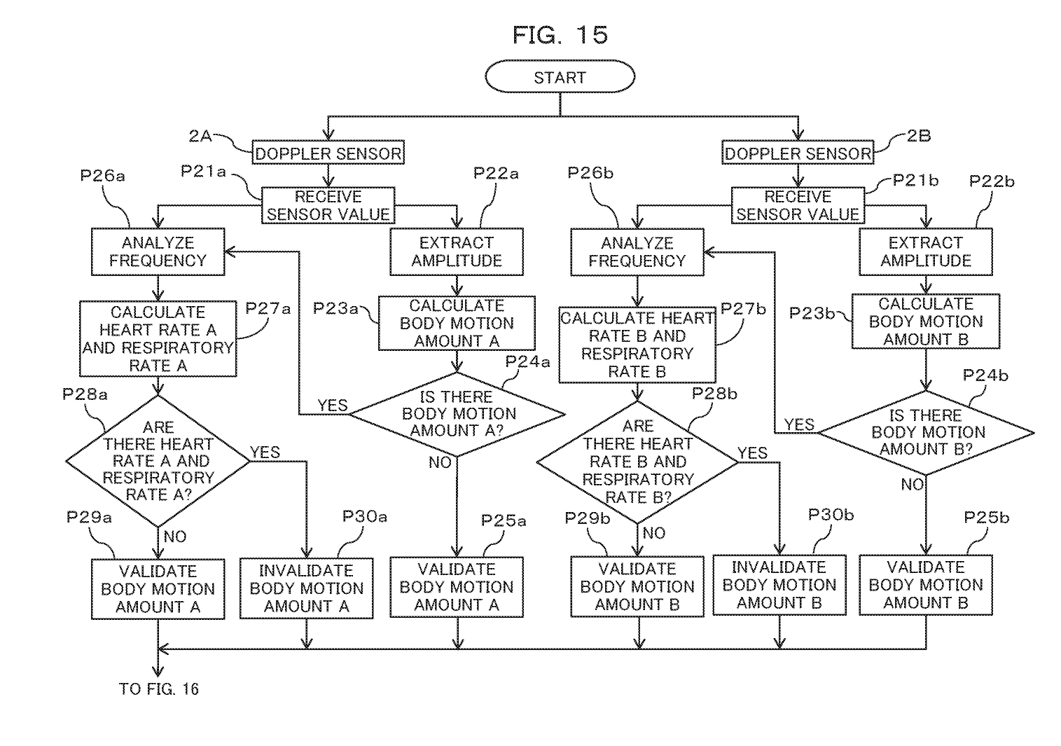

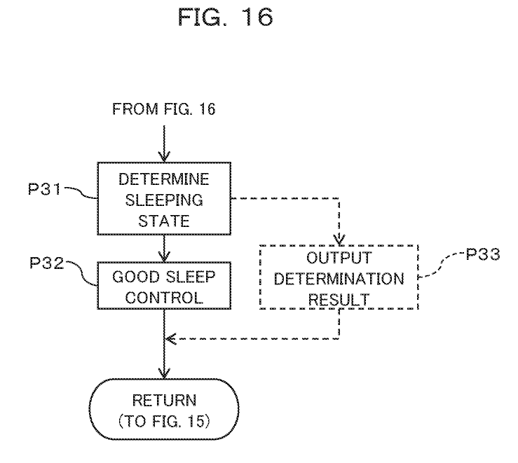

Next, a second embodiment of a process by the information processing apparatus 3 will be described with reference to FIGS. 15 and 16. Additionally, in the second embodiment, the configuration examples of the sensor system 1, the Doppler sensors 2A and 2B, and the information processing apparatus 3 may be the same as those of the first embodiment.

In the above-described first embodiment, the body motion amount of the user A or B has corrected in such a manner that the body motion amounts, the heart rates, and the respiratory rates of the users A and B are stored as DB in the DB 33 and the data is compared.

In the second embodiment, an example will be described in which the body motion amount of the user A or B is corrected by sequentially comparing the data without storing the body motion amounts, the heart rates, and the respiratory rates of the users A and B as DB in the DB 33.

By a sequential data comparing process, it is possible to suppress a delay in the body motion amount correction process and to further suppress a delay in the sleeping state determination process compared to the first embodiment. Thus, it is possible to improve the real time property of the sleeping state determination.

As illustrated in FIG. 15, the information processing apparatus 3 receives the Doppler sensor values A and B transmitted from the Doppler sensors 2A and 2B to the information processing apparatus 3 (Processes P21a and P21b). For example, the Doppler sensor values A and B are received by the communication IF 34 of the information processing apparatus 3 and are input to the processor 31 of the information processing apparatus 3.

Similarly to the first embodiment, the processor 31 may extract the amplitude components of the sensor values A and B (Processes P22a and P22b) and may calculate the body motion amounts A and B of the users A and B based on the extracted amplitude component (Processes P23a and P23b).

When the body motion amounts A and B are calculated, the processor 31 may determine whether the body motion detection exists by comparing, for example, the body motion amounts A and B with the determination threshold values (Processes P24a and P24b).

For example, when the body motion amount A (or B) is equal to or larger than the determination threshold value, it may be determined that the “body motion detection” exists. Then, when the body motion amount is smaller than the determination threshold value, it may be determined that the “body motion detection” does not exist. Additionally, the determination threshold value of the body motion amount A and the determination threshold value of the body motion amount B may be, for example, the same value.

When the “body motion detection” for the body motion amount A of the user A does not exist as the determination result (NO in Process P24a), the processor 31 may treat the body motion amount A of the user A as the valid data (Process P25a).

In other words, the processor 31 may process the body motion detection based on a change in amplitude of the Doppler sensor value A as the valid body motion detection. The process may be regarded as a process of detecting the body motion based on a change in amplitude of the Doppler sensor value A.

Similarly, when the “body motion detection” for the body motion amount B of the user B does not exist (NO in Process P24b), the processor 31 may process the body motion amount B of the user B as the valid data (Process P25b).

In other words, the processor 31 may process the body motion detection based on a change in amplitude of the Doppler sensor value B as the valid body motion detection. The process may be regarded as a process of detecting the body motion based on a change in amplitude of the Doppler sensor value B.

Meanwhile, when the “body motion detection” for the body motion amount A of the user A exists (YES in Process P24a), the processor 31 may analyze the frequency of the received sensor value A (Process P26a) and may calculate the heart rate A and the respiratory rate A of the user A (Process P27a).

Similarly, when the “body motion detection” for the body motion amount B of the user B exists (YES in Process P24b), the processor 31 may analyze the frequency of the received sensor value B (Process P26b) and may calculate the heart rate B and the respiratory rate B of the user B (Process P27b).

In the frequency analysis, FFT or DFT may be used similarly to the first embodiment. Further, the heart rate and the respiratory rate may be calculated similarly to the first embodiment.

Additionally, the frequency analysis of Process P26a (P26b) or the frequency analysis of Process P26a (P26b) and the calculation of the heart rate and the respiratory rate of Process P27a (27b) may be started regardless of the determination result of Process P24a (P24b).

When the heart rate A and the respiratory rate A of the user A are calculated, the processor 31 may determine whether the heart rate A and the respiratory rate A are calculated as appropriate values, in other words, one or both of the heart rate A and the respiratory rate A lack (Process P28a).

Similarly, when the heart rate B and the respiratory rate B of the user B are calculated, the processor 31 may determine whether the heart rate B and the respiratory rate B are calculated as appropriate values, in other words, one or both of the heart rate B and the respiratory rate B are lack (Process P28b).

Additionally, in the above-described determination, the determination threshold value may be used for each of the heart rate and the respiratory rate. For example, it may be determined that the heart rate exists when the heart rate is equal to or larger than the determination threshold value and it may be determined that the heart rate lacks when the heart rate is smaller than the determination threshold value. Similarly, it may be determined that the respiratory rate exists when the respiratory rate is equal to or larger than the determination threshold value and it may be determined that the respiratory rate lacks when the respiratory rate is smaller than the determination threshold value.

When the heart rate A and the respiratory rate A of the user A exist (in other words, both rates do not lack) (YES in Process P28a), the processor 31 may correct the body motion amount A of the user A to the invalid value (for example, 0) (Process P30a).

In other words, it is possible that the processor 31 does not process the body motion detection based on a change in amplitude of the Doppler sensor value A as the valid body motion detection. This is because the body motion amount A is caused by the body motion of the other user B instead of the body motion of the user A. The process may be regarded as a process of ignoring or not performing the body motion detection based on a change in amplitude of the Doppler sensor value A.

Meanwhile, when one or both of the heart rate A and the respiratory rate A of the user A lack (NO in Process P28a), the processor 31 may process (maintain) the body motion amount A of the user A as the valid data (Process P29a).

In other words, the processor 31 may process the body motion detection based on a change in amplitude of the Doppler sensor value A as the valid body motion detection. This is because the body motion amount A is caused by the body motion such as rolling motion of the user A. The process may be regarded as a process of detecting the body motion based on a change in amplitude of the Doppler sensor value A.

Also for the body motion amount B of the user B, the processor 31 may perform the above-described process for the user A.

For example, when the heart rate B and the respiratory rate B of the user B do not lack (YES in Process P28b), the processor 31 may correct the body motion amount B of the user B to the invalid data (for example, 0) (Process P30b).

In other words, it is possible that the processor 31 does not process the body motion detection based on a change in amplitude of the Doppler sensor value B as the valid body motion detection. This is because the body motion amount B is caused by the body motion of the other user A instead of the body motion of the user B. The process may be regarded as a process of ignoring or not performing the body motion detection based on a change in amplitude of the Doppler sensor value B.

Meanwhile, when one or both of the heart rate B and the respiratory rate B of the user B lack (NO in Process P28b), the processor 31 may process (maintain) the body motion amount B of the user B as the valid data (Process P29b).

In other words, the processor 31 may process the body motion detection based on a change in amplitude of the Doppler sensor value B as the valid body motion detection. This is because the body motion amount B is caused by the body motion such as rolling motion of the user B. The process may be regarded as a process of detecting the body motion based on a change in amplitude of the Doppler sensor value B.

After the above-described process, as illustrated in FIG. 16, the processor 31 may determine the sleeping states of the users A and B (Process P31). The determination of the sleeping state may be the same as that of the first embodiment. Here, also in the second embodiment, when the body motion occurs due to the rolling motion of one of the users A and B, the other body motion detection is not processed as the valid body motion detection.

Thus, since the possibility of the false detection that a person who is not moving has moved is reduced similarly to the first embodiment, it is possible to improve the body motion detection accuracy and to improve the sleeping state determination accuracy. Further, in the second embodiment, since the data of the body motion amounts, the heart rates, and the respiratory rates of the users A and B is sequentially compared as described above, it is possible to suppress a delay in the body motion amount correction process and a delay in the sleeping state determination process compared to the first embodiment. Thus, it is possible to improve the real time property of the sleeping state determination.

Additionally, in the second embodiment, as illustrated in FIG. 16, the processor 31 may perform “good sleep control” which helps the good sleep of the users A and B by controlling the operation of the air conditioner 7 or the luminaire 8 based on the sleeping state determination result similarly to the first embodiment (Process P32). Further, the processor 31 may appropriately output the sleeping state determination result to an external device such as a display or a printer as a report or the like similarly to the first embodiment (Process P33).

The second embodiment is also the same as the first embodiment in that three or more persons can use one bed 5.

### Third Embodiment

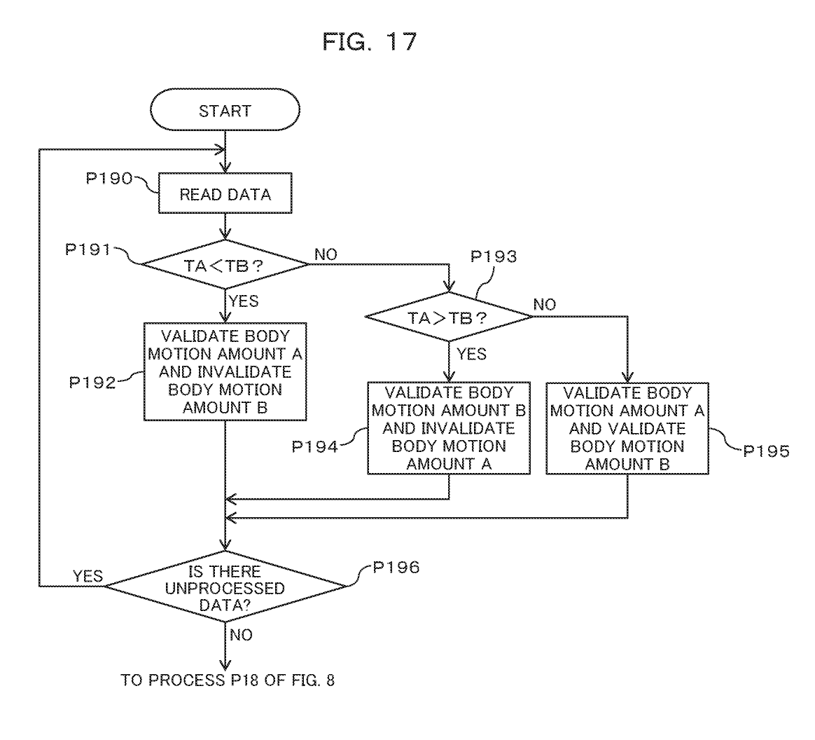

Next, a third embodiment of a process by the information processing apparatus 3 will be described with reference to the flowchart illustrated in FIG. 17. Additionally, in the third embodiment, the configuration examples of the sensor system 1, the Doppler sensors 2A and 2B, and the information processing apparatus 3 may be the same as those of the first embodiment and the second embodiment.

It may be understood that the flowchart illustrated in FIG. 17 corresponds to the modified example of the flowchart illustrated in FIG. 9. In other words, in the third embodiment, the processor 31 of the information processing apparatus 3 may perform the flowchart illustrated in FIG. 17 in Process P17 for correcting the body motion amount illustrated in FIG. 8.

In the first embodiment and the second embodiment described above, the heart rate and the respiratory rate calculated by analyzing the frequencies of the Doppler sensor values A and B are used to determine whether the body motion such as rolling motion occurs in any one of the users A and B.

In the third embodiment, it is determined that the body motion occurs in the user corresponding to the sensor value indicating that the body motion such as rolling motion occurs first in time regardless of the heart rate and the respiratory rate. For example, in the body motion amounts A and B detected at the same time period based on the sensor values A and B, the body motion amount A (or B) detected at the earliest timing is validated and the other body motion amount B (or A) is invalidated.

In the third embodiment, since the body motion amount of the user estimated that the body motion does not occur can be invalidated without using the frequency analysis result of the Doppler sensor values A and B as in the first embodiment or the second embodiment, it is possible to simplify the process compared to the first embodiment and the second embodiment.

As illustrated in FIG. 17, the processor 31 may read data by referring to the DB 33 (Process P190) and may compare the timings TA and TB of the body motion amounts A and B detected to be shifted from each other at a same time period (Process P191).

When the timing TB is earlier than the timing TA as the comparison result (YES in Process P191), the processor 31 may maintain the body motion amount A as the valid data and may correct the body motion amount B to the invalid value (for example, 0) (Process P192).

In other words, the processor 31 may determine a Doppler sensor which could obtain the sensor value corresponding to the body motion indicating the first rolling motion as the Doppler sensor 2A and may process the body motion detection based on a change in amplitude of the Doppler sensor value A as the valid body motion detection. On the contrary, it is possible that the processor 31 does not process the body motion detection based on a change in amplitude of the Doppler sensor value B as the valid body motion detection.

Meanwhile, when the relationship of TA<TB is not established (NO in Process P191), the processor 31 may further determine whether the relationship of TB<TA is established (Process P193). When the relationship of TB<TA is established (YES in Process P193), the processor 31 may maintain the body motion amount B as the valid data and may correct the body motion amount A to the invalid value (for example, 0) (Process P194).

In other words, the processor 31 may determine a Doppler sensor which could obtain the sensor value corresponding to the body motion indicating the first rolling motion as the Doppler sensor 2B and may process the body motion detection based on a change in amplitude of the Doppler sensor value B as the valid body motion detection. On the contrary, it is possible that the processor 31 does not process the body motion detection based on a change in amplitude of the Doppler sensor value A as the valid body motion detection.

When neither TA<TB nor TB<TA (NO in Processes P191 and P193), the relationship of TA=TB is established and thus the processor 31 may maintain both of the body motion amounts A and B as the valid data (Process P195).

In other words, the processor 31 may process the body motion detection based on a change in amplitude of the Doppler sensor values A and B as the valid body motion detection.

The processor 31 may repeat the process after Process P190 (YES in Process P196) until no unprocessed data is left in the DB 33 (until No in Process P196).

When no unprocessed data is left (NO in Process P196), the processor 31 may determine the sleeping states of the users A and B, for example, based on the data of the body motion amounts, the heart rates, and the respiratory rates of the users A and B registered in the DB 33 as illustrated in FIG. 8 (Process P18 in FIG. 8).

The determination of the sleeping state may be the same as that of the first embodiment. Here, also in the third embodiment, when the body motion such as rolling motion occurs in one of the users A and B, the other body motion detection is not processed as the valid body motion detection.

Thus, since the possibility of the false detection that a person who is not moving has moved is reduced similarly to the first embodiment and the second embodiment, it is possible to improve the body motion detection accuracy and to improve the sleeping state determination accuracy.

Further, in the third embodiment, since it is possible that the frequency analysis result of the Doppler sensor values A and B is not used for the body motion amount correction process as described above, it is possible to simplify the body motion amount correction process compared to the first embodiment and the second embodiment. Thus, it is possible to reduce the processing amount of the processor 31. In other words, it is possible to reduce the processing capacity demanded for the processor 31.

Additionally, also in the third embodiment, as illustrated in FIG. 8, the processor 31 may perform “good sleep control” which helps the good sleep of the users A and B by controlling the operation of the air conditioner 7 or the luminaire 8 based on the sleeping state determination result similarly to the first embodiment (Process P19 of FIG. 8). Further, the processor 31 may appropriately output the sleeping state determination result to an external device such as a display or a printer as a report or the like similarly to the first embodiment (Process P20 of FIG. 8).

Also in the third embodiment, it is possible to handle a case in which three or more persons use one bed 5. For example, the body motion amount obtained based on the sensor value at the first timing illustrating the body motion detection in the determination time period among the sensor values of three or more Doppler sensors 2 may be validated and the body motion amount obtained based on the other sensor value may be invalidated.

### Comparative Example

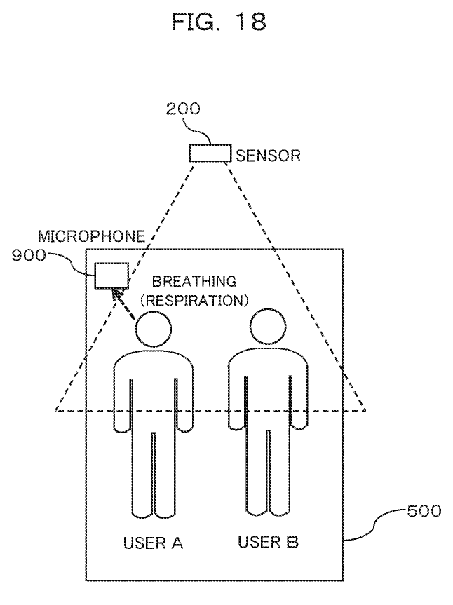

FIG. 18 illustrates a comparative example of an embodiment including the first to third embodiments. FIG. 18 illustrates an example in which one Doppler sensor 200 and a microphone 900 are installed in an indoor space in a case in which two users A and B sleep on one bed 500 side by side.

The Doppler sensor 200 is installed in, for example, a ceiling or a wall of the indoor space so that the chests of the users A and B are included in the sensing range. In other words, the Doppler sensor 200 is common to the users A and B differently from the above-described embodiments.

The microphone 900 is installed at a position capable of collecting the breathing sound of one of the users A and B, for example, at a bedside or the like of the user A (or B).

In the example of FIG. 18, when the frequency of the sensor value of the Doppler sensor 200 is analyzed, it is possible to obtain the frequency signal in which the heartbeat components and the respiration components of two users A and B are mixed with each other.

However, even when a combination of the heartbeat component and the respiration component of the same user A or B can be specified from the frequency signal, it is difficult to specify the heartbeat component and the respiration component for any one of the users A and B.

Here, it is possible specify a combination of the heartbeat component and the respiration component of one user A (or B) by analyzing the breathing sound of one user A (or B) collected by the microphone 900.

Many users care about privacy about the microphone 900 installed in an indoor space such as a bedroom. Further, when the position of the bed 500 is changed, the installation position of the Doppler sensor 200 needs to be changed according to the change and thus the degree of freedom in the arrangement relationship between the Doppler sensor 200 and the bed 500 is low.

On the contrary, according to the above-described embodiments, since it is possible that the microphone 900 is not installed in the indoor space, the privacy of the user can be protected. Further, since the Doppler sensors 2A and 2B are attached to the bed 5, the positional relationship between the users A and B corresponding to the sensing ranges of the sensors 2A and 2B is not changed even when the arrangement position of the bed 5 is changed. Thus, it is possible to improve the degree of freedom in the arrangement position of the bed 5 in the indoor space.

Additionally, in the example of FIG. 18, since a change in amplitude corresponding to the motion of the users A and B is mixed in the sensor value of one Doppler sensor 2 in the same frequency band, it is difficult to separate the “body motion” of the users A and B even if the frequency analysis is performed.

On the contrary, according to the above-described embodiments, it is possible to highly accurately detect whether the body motion of the plurality of users exist in a non-contact manner by using the plurality of Doppler sensors and thus to improve the sleeping state estimation accuracy based on the body motion.

(Others)

Additionally, in the embodiments including the above-described embodiments, an example in which the information processing apparatus 3 receives the sensor values of the sensors 2A and 2B via the network 4 has been described. However, the information processing apparatus 3 may be provided in, for example, the indoor space provided with the bed 5 and may receive the sensor values without using the network 4.

According to the above-described technologies, it is possible to improve body motion detection accuracy for a plurality of persons using a plurality of Doppler sensors.

All examples and conditional language provided herein are intended for pedagogical purposes to aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiment(s) of the present invention have been described in detail, it should be understood that the various changes, substitutions, and alterations could be made hereto without departing from the spirit and scope of the invention.

What is claimed is:

1. A sensor system comprising:
a transmitter configured to transmit radio wave;
a receiver configured to receive a wave corresponding to the radio wave from the transmitter; and
a detector configured to detect a body motion when a change in amplitude of the wave received by the receiver is detected and a lack of a frequency component indicating at least one of a heartbeat and a respiration in a frequency analysis result of the wave is detected.

2. The sensor system according to claim 1, wherein
the detector is configured to control not to process a detection of the body motion based on the change in amplitude of the wave as a valid body motion detection when the lack of the frequency component is not detected.

3. A sensor system comprising:
a plurality of Doppler sensors configured to be arranged at different positions of a bed respectively; and
a processor configured to:
acquire a plurality of sensor values from the plurality of Doppler sensors, and
detect a body motion based on a sensor value, from among a part of the plurality of sensor values, in which a lack of a frequency component indicating at least one of a heartbeat and a respiration is detected in a frequency analysis result for the plurality of sensor values, the part of the plurality of sensor values indicating a change in amplitude.

4. The sensor system according to claim 3, wherein
the processor is configured to control not to process the detection of the body motion based on the sensor value in which the lack of the frequency component is not detected as a valid body motion detection.

5. The sensor system according to claim 1, wherein
the processor is configured to determine a sleeping state based on the body motion detection result and the frequency analysis result.

6. A bed comprising:
a first Doppler sensor configured to sense a sensing range by a radio wave, the sensing range including a part or all of a first sleeping region of the bed; and
a second Doppler sensor that configured to sense another sensing area by the radio wave, the other sensing range including a part or all of a second sleeping region of the bed, the second sleeping region being different from the first sleeping region.

7. The bed according to claim 6, wherein
one of the first and second sleeping regions is a left region of the bed in a width direction and the other of the first and second sleeping regions is a right region of the bed in the width direction.

8. The bed according to claim 6, wherein
the bed has a width equal to or wider than a width of a double bed.

9. The bed according to claim 8, wherein
the width of the double bed is 1400 mm.

10. The bed according to claim 6, wherein
the bed is a double bed.

11. The bed according to claim 6, wherein
the first and second Doppler sensors are configured to be attached to a floor plate on which a mattress is placed in the bed.

12. The bed according to claim 6, wherein
the first and second Doppler sensors are configured to be attached to a headboard of the bed.

13. The bed according to claim 6, wherein
one of the first and second Doppler sensors is configured to be attached to a floor plate on which a mattress is placed in the bed, and
the other of the first and second Doppler sensors is configured to be attached to a headboard of the bed.
